(19) United States
(12) Patent Application Publication
(10) Pub. No.: US 2006/0284732 A1
(43) Pub. Date: Dec. 21, 2006

(54) Heart monitor with remote alarm capability

(76) Inventor: George Brock-Fisher, Andover, MA (US)

Correspondence Address: PHILIPS INTELLECTUAL PROPERTY & STANDARDS, 595 MINER ROAD, CLEVELAND, OH, 44143, US

(21) Appl. No.: 10/595,243

(22) PCT Filed: Oct. 6, 2004
(86) PCT No.: PCT/IB04/52048

§ 371 Date: Mar. 29, 2006

Related U.S. Application Data
(60) Provisional application No. 60/513,629, filed on Oct. 23, 2003.

(51) Int. Cl. G08B 23/00; A61B 5/00

(52) U.S. Cl. 340/573.1; 600/300

(57) ABSTRACT

A mobile phone (108) is configured as an adjunct to a medical monitoring device (100) to make an emergency phone call (228) to a predetermined, trusted care-giver or -overseer (120) when an abnormal medical condition is detected by the device (204). The phone and the device preferably communicate wirelessly with each other, as through a Bluetooth connection (212). Preferably, the phone is equipped to play back a pre-recorded voice message on the phone call (236), which may include the current location of the patient (216, 220), instructions personalized to the care-giver (168), and an image or video playback of the patient. The phone may concurrently page emergency medical services.

The present invention relates to medical monitoring and notification in the event of an emergency. More particularly, the present invention is directed to mobile means for both the monitoring and notification.

Heart disease is a leading killer. Often, a fatal heart attack is not preceded by any symptoms that are noticeable or that have been noticed. In other cases, a person may be aware that he or she has heart disease and may wish to prepare so that an abnormal heart condition is detected and help is summoned immediately.

U.S. Pat. No. 6,579,231 to Phipps, entitled “Personal Medical Monitoring Unit and System,” the entire disclosure of which is hereby incorporated herein by reference, describes a mobile medical monitoring unit designed to be worn by the patient. When the unit detects an adverse medical condition of the patient, it sends out a wireless signal to a communications network which relays the alarm to a 911 call center and a central reporting system storing instructions for further responding to the emergency. The unit front-end is a monitor device and the back-end is a transmitter.

Phipps mentions that it is preferable to implement the notifying transmitter as a pager, rather than as a portable phone, due to the smaller form factor which, importantly, increases the mobility of the device. This, however, limits one's choice as to the receiver, since the receiver must subscribe to a service provider to be able to receive the page and must utilize a device for receiving the page. Moreover the chosen receivers must have the facilities to receive the entire page reliably and quickly to effectively respond to the emergency.

Similarly, for the Phipps portable phone implementation, since it operates by transmitting over data channels, the receiver must have available the facilities to receive and interpret this data. This, again, limits the choice of responders.

In addition, for either implementation, the designated receivers of the emergency notification may not be located as close to the victim as others who could arrive quicker. Portable, commercially-available defibrillators, for example, that are user-friendly are now becoming more widespread and could be brought to the victim by a close friend or relative in the immediate vicinity.

Another problem is that the patient may want a personally close individual to handle or oversee the handling of the emergency, but may not want to worry that individual by informing him or her in advance of the heart disease or its current state.

What is needed is an apparatus that is easily mobile and which affords quick, reliable, automatic and personal communicating of the medical emergency to designated individuals without having to rely solely on specialized services.

The present invention has been made to address the above-noted shortcomings in the prior art and, in one aspect, affords a personalized way of notifying designated, trusted individuals of a medical emergency.

In another aspect, those in close proximity of the victim can be notified.

In yet another aspect of the invention, the present invention leverages existing communication infrastructure, and in portable wireless phones which enjoy widespread usage, to increase the mobility of a portable medical monitoring apparatus, and therefore the willingness of patients to wear the apparatus. Use of existing facilities also decreases the size and cost of the monitoring apparatus.

These and other aspects, features and advantages of the present invention are realized in a portable medical monitoring apparatus that includes a portable monitoring device for medically monitoring to detect the occurrence of a predefined abnormal condition. Attached to the device is a wireless transmitter that automatically, wirelessly and directly communicates the occurrence, upon its detection, to a portable phone. At that point, the phone makes an emergency call.

In a further embodiment, a portable medical monitoring system includes a portable monitoring apparatus for medically monitoring to detect the occurrence of a predefined abnormal condition. Upon detection of the medical condition, an automatic dialer in the phone calls a particular phone number. When the call is answered, the phone plays a pre-recorded message on the call.

Details of the invention disclosed herein shall be described with the aid of the figures listed below, wherein:

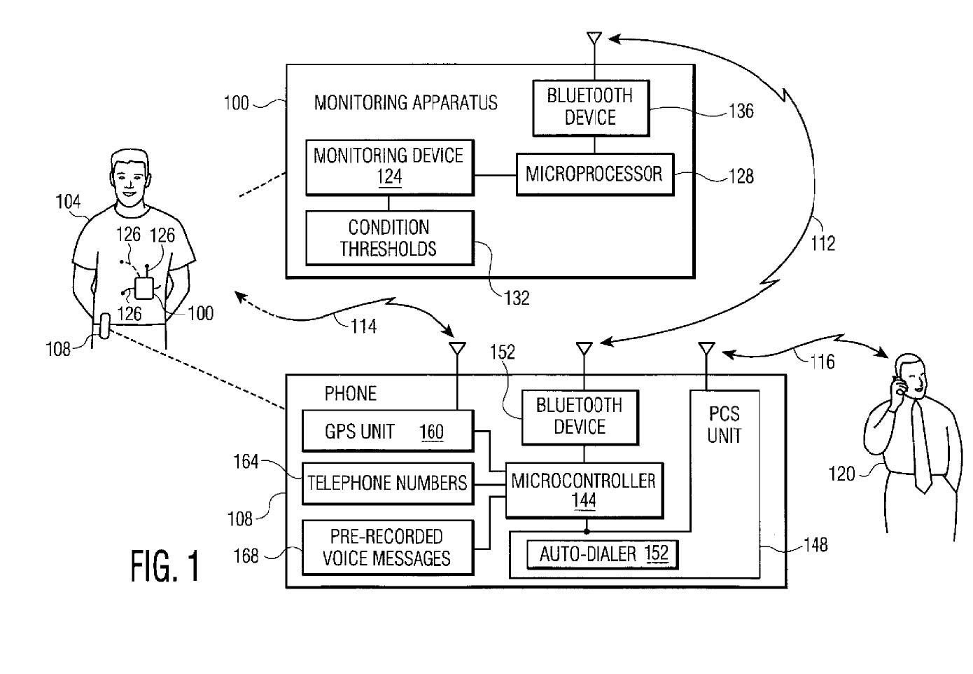

FIG. 1 is a conceptual block and flow diagram of an exemplary portable medical monitoring apparatus in accordance with the present invention; and

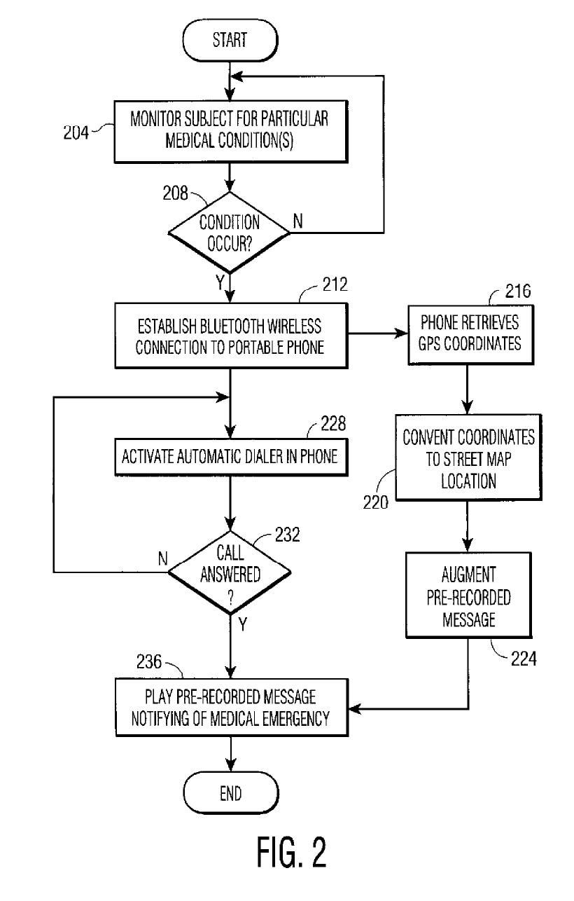

FIG. 2 is a flow chart illustrating an example of a process performed by the apparatus depicted in FIG. 1.

FIG. 1 is a conceptual block and flow diagram of an exemplary portable medical monitoring apparatus 100 in accordance with a preferred embodiment of the present invention. Detailed description of known elements and features has been omitted for clarity of presentation.

In FIG. 1, a patient 104 is wearing the apparatus 100, and attached to the patient's clothing is a portable phone 108. Extending from each of the apparatus 100 and phone 108 are respective broken lines leading to more detailed views of the devices in block diagram form. A wireless signal 112 from the apparatus 100 to the phone 108 reports the emergency, a wireless signal 114 from a satellite (not shown) provides the present location of the phone, and a responsive wireless signal 116 from the phone originates a phone call to a trusted recipient 120 who is to provide or summon aid.

The monitoring apparatus 100 includes a monitoring device 124 which may be a microprocessor and, in the implementation shown, is an electrocardiogram (EKG) unit, such as that described in U.S. Pat. No. 4,546,776 to Bellin et al., entitled “Portable EKG Monitoring Device For ST Deviation,” the disclosure of which is incorporated by reference in its entirety. The EKG unit 124 is worn by the patient 104, as by means of a patch underneath the clothing, with electrodes 126 extending to the appropriate monitoring points on the body of the patient 104. Alternatively, the portable medical monitoring can be implemented by means of ultrasound, acoustic waves or by other known and suitable means. Nor is the intended scope of the invention limited to cardiac monitoring, but may extend to other types of medical monitoring such as blood sugar level. Moreover, and as mentioned in the above-described Phipps patent, although the present invention is directed primarily to those known to have medical conditions, the monitoring may be utilized merely, for example, to provide early warning of potentially developing medical problems.

The monitoring device is connected to a microprocessor 128 and to a memory 132 such as a random access memory (RAM) or a read-only memory (ROM) for storing thresholds pertaining to specific medical conditions. The microprocessor 128 interfaces with a Bluetooth device 136. Bluetooth is an emerging technology for mobile, wireless communication among two or more devices that incorporate respective Bluetooth devices, the devices being located within a range of about 10 meters. Increasingly, Bluetooth devices are utilized in mobile phones, for example.

The portable phone 108, such as a mobile, cellular or personal communications system (PCS) phone, features a microcontroller 144 in communication with a PCS unit 148 having an automatic dialer or auto-dialer 152 like those typically utilized in cell phones for voice dialing, speed dialing or automatic redial. Also interfaced to the microcontroller 144 is a Bluetooth device 156, a global positioning system (GPS) unit 160, a memory 164 for storing one or more telephone numbers and a memory 168 for storing one or more pre-recorded voice messages. The memories 164, 168 may be implemented in RAM or ROM in any of their various forms.

FIG. 2 is a flow chart that demonstrates, by way of non-limitative example, processes performed by the monitoring apparatus 100 and the phone 108. The monitoring device 124 monitors the patient 104 to detect one or more abnormal conditions based on respective thresholds or criteria stored in memory 132 (step 204). Upon occurrence of an abnormal condition (step 208), the microprocessor 128 signals the Bluetooth device 136 of the monitoring apparatus 100 to establish a wireless Bluetooth connection with the respective Bluetooth device 156 of the phone 108 (step 212). The Bluetooth device 156 of the phone 108 proceeds to complete the Bluetooth connection over which an alarm is sent to the Bluetooth device 156.

The alarm is passed on to the microcontroller 144 which then commands the GPS unit 160 to determine the current location of the phone 108 (step 216). The GPS unit 160 preferably has mapping information to translate GPS coordinates received from a GPS satellite to a street address (step 220), e.g., two intersecting streets, and preferably has a speech synthesizer for creating a voice signal that, when played back, recites the street address. The GPS unit may further include logic for associating a location label such as “home” with one or more particular street addresses, so that the voice signal may instead or additionally recite that label.

The microcontroller also retrieves from storage 168 one or more pre-recorded messages having been prepared in advance for storage in RAM 168, and have been entered into storage by, for example, the patient 104 speaking into the microphone (not shown) of the portable unit 108 while activating or having activated the appropriate key(s) on the keyboard (not shown). The pre-recorded message may have a gap, or silent portion, into which the voice signal telling the patient's location is inserted, or, alternatively, the voice signal may be appended to the pre-recorded message (step 224). If the recipient of the alarm has a suitably equipped phone, e.g. with the screen of a typical camera phone, the pre-recorded message may be accompanied by an image or video signal portraying the patient 104, with the pre-recorded message starting out as, for example, “Jim, its me Jeff. Look quickly at the phone screen and then listen.” The goal is to establish a personal connection with the care-giver or care-summoner 120.

Preferably concurrently with involving the GSP unit 160, the microcontroller 144 retrieves from storage 164 one or more telephone numbers associated with the pre-recorded message(s). The microcontroller 144 notifies the PCS unit 148 of the alarm and passes on the telephone number(s) so that the automatic dialer 152 can make the predetermined call(s) (step 228). Any known and suitable means may be incorporated into the phone 108 to detect if the call is being answered by an answering machine or voice mail, so that, in such an event, the phone 108 can “hang up” and make an immediate call to a second, stored telephone number. The phone 108 may be implemented additionally for wireless paging to contact, for example, a 911 Call Center as described in the Phipps patent. If the call is personally answered (step 232), the augmented, pre-recorded message is played back over the call to notify the trusted recipient 120 of the medical emergency and the current location of the patient 104 (step 236). The pre-recording may include instructions tailored to the particular recipient 120, and may be accompanied by an image or video playback of the patient 104.

Although the monitoring apparatus 100 and the phone 108 are depicted as Bluetooth-enabled, it is within the intended scope of the invention that their connection may be wired as in the Phipps patent. Also, although voice channels are used in the preferred embodiment described above, data channels may instead, or in addition, be employed to deliver text data to a phone of the trusted care-giver 120 or, as discussed in Phipps, to other destinations from which emergency help may be summoned.

As set forth above, the present invention affords a personalized way of providing notification of a medical emergency to designated, trusted individuals, and particularly those in close proximity to the victim. The facilities and infrastructure for portable, wireless technology in widespread usage are used to advantage as an alternative or supplement to emergency call centers and as a means for reducing the overall form factor, thereby increasing a patient's willingness to wear the apparatus and the cost of the apparatus.

While there have been shown and described what are considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. A portable medical monitoring apparatus comprising:
a portable monitoring device for medically monitoring to detect an occurrence of a predefined abnormal condition; and
a wireless transmitter attached to the device and configured for automatically wirelessly and directly communicating the occurrence to a portable phone upon detection by the device of said occurrence, the phone being configured to make an emergency call upon receiving the communication.

2. The apparatus of claim 1, wherein the monitorIng is of a subject, the device being attached to the subject during the monitoring.

3. The apparatus of claim 1, wherein the phone comprises a Bluetooth device, said apparatus further comprising a Bluetooth device configured for said communicating with the Bluetooth device of the phone.

4. The apparatus of claim 1, further comprising the phone, said phone having an automatic dialer, the phone being configured to, upon said receiving the communication, activate the automatic dialer to call a particular phone number.

5. The apparatus of claim 4, wherein the phone is further configured to play a pre-recorded message on the call when the call is answered.

6. A portable medical monitoring system comprising:
a portable monitoring apparatus for medically monitoring to detect an occurrence of a predefined abnormal conditions; and
a portable phone having an automatic dialer and configured to, upon detecting of said occurrence by said apparatus, activate the automatic dialer to call a particular phone number, the phone being further configured to play a pre-recorded message on the call when the call is answered.

7. The system of claim 6, wherein the apparatus is configured to communicate said detecting of said occurrence automatically, wirelessly and directly to the phone.

8. The system of claim 6, wherein the monitoring is of a subject, the device being attached to the subject during the monitoring.

9. The system of claim 6, wherein the phone comprises a Bluetooth device, said apparatus further comprising a Bluetooth device configured for communicating to the Bluetooth device of the phone said detecting of said occurrence.

10. The system of claim 6, wherein the phone further comprises a global positioning system (GPS) unit configured for determining a street map location of the phone, the phone being further configured to augment the pre-recorded message with said locations.

11. The system of claim 6, wherein the pre-recorded message is a voice message and the playing of the message occurs on a voice channel.

12. A portable medical monitoring method comprising the steps of:
medically monitoring, with a portable monitoring device, to detect an occurrence of a predefined abnormal conditions;
automatically, wirelessly and directly communicating, by a wireless transmitter attached to the device, the occurrence to a portable phone upon detection by the device of said occurrence; and
making, by said phone, an emergency call upon receiving the communication.

13. The method of claim 12, wherein the call making step further comprises the step of activating an automatic dialer in the phone to call a particular phone number.

14. The method of claim 12, wherein call making step comprises the step of playing by said phone, a pre-recorded message on the call when the call is answered.

15. A portable medical monitoring method comprising:
medically monitoring, by a portable medical monitoring apparatus, to detect an occurrence of a predefined abnormal condition;
upon detecting of said occurrence by said apparatus, activating all automatic dialer in the phone to call a particular phone number; and
playing, by said phone, a pre-recorded message on the call when the call is answered.

16. The method of claim 15, further comprising the step of automatically, wirelessly and directly communicating, by said apparatus, to the phone said detecting of said occurrence.

17. The method of claim 15, wherein the monitoring is of a subject, the device being attached to the subject during the monitoring.

18. A portable medical monitoring apparatus comprising:
a portable monitoring device for monitoring one or more medical conditions for the occurrence of one or more predefined abnormal conditions;
a portable communication device for receiving a signal from the portable monitoring device that one of the predefined abnormal conditions has occurred;
wherein the portable communication device relays the occurrence of the predefined abnormal condition to one or more selected emergency contacts by providing a notification of the occurrence of the abnormal condition that has occurred and a location of the of the portable monitoring device.

19. The portable medical monitoring apparatus of claim 18, wherein the location is defined by a global positioning system.

20. The portable medical monitoring apparatus of claim 18, wherein selected locations can be predefined with location labels.

21. The portable medical monitoring apparatus of claim 18, wherein the emergency contact that is closest to the location is notified first.

22. The portable medical monitoring apparatus of claim 18, wherein the notification comprises a telephone to the one or more emergency contacts including a selected, pre-recorded message.
